(19) United States
(12) Patent Application Publication
(10) Pub. No.: US 2007/0056591 A1
(43) Pub. Date: Mar. 15, 2007

(54) Fallopian tube occlusion devices and methods

(76) Inventor: Hugh McSwain, San Francisco, CA (US)

Correspondence Address: MANUEL F. DE LA CERRA, 6885 CATAMARAN DRIVE, CARLSBAD, CA, 92011, US

(21) Appl. No.: 11/227,873

(22) Filed: Sep. 15, 2005

(51) Int. Cl. A61F 6/06

(52) U.S. Cl. 128/831

(57) ABSTRACT

A contraceptive device for placement in a fallopian tube includes expanding distal and proximal anchor members and an expandable elongate member connecting the distal anchor member to the proximal anchor member. An expandable material is disposed on at least a portion of the contraceptive device, whereupon delivery of the contraceptive device, the expandable material expands to completely occlude the fallopian tube. The expandable material may contain a drug, therapeutic agent, or hormone which is released over time to occlude or otherwise prevent the passage of spermazoa through the fallopian tube, or prevent ovulation. The contraceptive device may be delivered non-operatively and provide complete sterilization within a period of days. The device and delivery method obviates the need for follow-up visits to confirm closure of the fallopian tubes.

### FIELD OF THE INVENTION

The field of the invention generally relates to female contraceptive devices and methods. More specifically, the field of invention pertains to the intrafallopian contraceptive devices and non-surgical methods of delivery.

### BACKGROUND OF THE INVENTION

There is a significant demand for devices and methods that provide for safe and effective methods of permanent sterilization and contraception. With respect to permanent sterilization, bilateral tubal sterilization (BTS) is safe and effective for use as a contraceptive. BTS is currently the “gold standard” in permanent sterilization in female patients. For example, in the United States alone, approximately 11 million women rely on BTS for contraception with an estimated 700,000 procedures performed annually—making it the most common contraceptive method in the country.

BTS is performed almost exclusively by operative tubal ligation (bilateral tubal ligation or BTL). Surgical approaches for BTL include laparoscopy, microlaparoscopy, laparotomy (concurrent with cesarean delivery), minilaparotomy, and vaginal approaches. Because of the surgical nature of BTL, the procedure carries all the risks associated with operative intervention and anesthesia. Attempts have been made to provide a non-operative method of BTS. For example, the ESSURE micro-insertion device made by Conceptus, Inc. of San Carlos, Calif., is placed hysteroscopically and has been established as a safe and effective procedure for transcervical tubal sterilization (TTS). Also, Adiana, Inc. of Redwood City, Calif., has developed a hysteroscopically-placed device which uses low level radiofrequency energy to damage the fallopian tubes. A small plug is left behind in the tube to facilitate closure.

Unfortunately, devices such as the ESSURE micro-insertion device or the Adiana device do not provide immediate or near immediate contraceptive abilities. For example, with respect to the ESSURE device, there is a waiting period of at least three months after placement to ensure that the device is efficacious as a contraceptive. Patients receiving the ESSURE device are required to follow-up with their physician for, among other things, a hysterosalpingogram (HSG) to confirm complete tubal occlusion. During this interim waiting period, alternative birth control methods must be used by the patient. This is in contrast with conventional BTL procedures wherein permanent sterilization is established as soon as the patient recovers from surgery.

There thus is a need for a permanent tubal sterilization device which uses a non-surgical method of delivery and provides immediate or near-immediate contraceptive efficacy. Such a device and method would offer the significant advantage of avoiding the lengthy waiting period that accompanies current devices such as the ESSURE. The device and method would offer the non-surgical benefits of devices like the ESSURE as well as the immediate contraceptive efficacy of conventional BTL.

### SUMMARY OF THE INVENTION

The fallopian tube occlusion device and method described herein provide both short-term and long-term occlusion of the fallopian tube to provide a safe and effective permanent sterilization method. The occlusion devices and methods described herein obviate the need for follow-up visits to the physician or other health care provider to confirm occlusion of the fallopian tube(s).

In a first embodiment, a contraceptive device for placement in a fallopian tube includes expanding distal and proximal anchor members and an expandable elongate member connecting the distal anchor member to the proximal anchor member. An expandable material is disposed on at least a portion of the contraceptive device, whereupon delivery of the contraceptive device, the expandable material expands to substantially or completely occlude the fallopian tube. The contraceptive device may be delivered non-operatively and provide complete sterilization within a period of days. The device and delivery method obviates the need for follow-up visits to confirm closure of the fallopian tubes.

In another embodiment, an apparatus for placement of a contraceptive device in a fallopian tube includes a delivery member having a lumen passing from a proximal end to a distal end, a contraceptive device adapted for disposal within the lumen of the delivery member, an expandable material disposed on at least a portion of the contraceptive device, and a pusher member slidable within the lumen of the delivery member, the pusher member being located proximal of the contraceptive device. The contraceptive device includes expanding distal and proximal anchor members and an expandable elongate member connecting the distal anchor member to the proximal anchor member. An expandable material is disposed on at least a portion of the contraceptive device, whereupon delivery of the contraceptive device, the expandable material expands to substantially or completely occlude the fallopian tube.

In one aspect of the invention, the expandable material may be made of an expandable hydrogel. The hydrogel or other expandable material may be loaded with a drug or therapeutic agent such as, for example, a spermicidal agent, an anti-viral agent, an anti-microbial agent, a sclerosing agent, and/or an inflammatory agent. The inflammatory agent may include synthetic materials or naturally occurring substances such as cytokines which may induce inflammation, cellular proliferation and/or cellular migration.

In one aspect of the invention, the fallopian tube is completely occluded within about four days after delivery of the contraceptive device. The expandable material may degrade or be absorbed by the body over the following weeks or months. The fallopian tube, however, still remains occluded during the degradation of the expandable material because of low-level, long term fibrosis and/or inflammation of the fallopian tube.

In another aspect of the invention, a method of occluding a fallopian tube is provided and includes the steps of providing a contraceptive device and inserting the contraceptive device into the fallopian tube, whereupon insertion, one or both of distal and proximal anchor members expand to secure the contraceptive device to an interior surface of the fallopian tube. An expandable material disposed on at least a portion of the contraceptive device is expanded to completely occlude the fallopian tube.

In one aspect of the invention, the fallopian tube is completely occluded within about four days after delivery of the contraceptive device. The expandable material may be loaded with a drug or therapeutic agent that is eluted or released over a period of time. The expandable member may be degraded and/or absorbed by the body over a period of days, weeks, or months. The fallopian tube(s) remain occluded, however, because of secondary fibrosis and/or inflammation of the fallopian tube(s).

It is an object of the invention to provide a intrafallopian contraceptive device and non-surgical method of delivery. The contraceptive device, once deployed, provides immediate or near immediate contraceptive abilities. To this end, it is a further object of the invention to provide a contraceptive device and method that does not require follow-up visits to a physician or other health care professional to confirm occlusion of the fallopian tube(s). Additional objects of invention are discussed below with reference to the drawings and the description of the preferred embodiments.

### BRIEF DESCRIPTION OF THE DRAWINGS

FIG. 1 illustrates a perspective view of contraceptive device for placement in a fallopian tube according to one embodiment.

FIG. 2 is a magnified cross-sectional view of the contraceptive device taken along the line A-A in FIG. 1.

FIG. 3 is a side cross-sectional view of delivery member and pusher member delivering a contraceptive device out a distal end thereof.

FIG. 4 is a cross-sectional view of a fallopian tube illustrating the deployed contraceptive device. The expandable material is shown in its fully expanded state.

FIG. 5 illustrates a perspective view of contraceptive device for placement in a fallopian tube according to another embodiment.

FIG. 6 is a magnified cross-sectional view of the contraceptive device taken along the line B-B in FIG. 5.

FIG. 7 is a side cross-sectional view of delivery member and pusher member delivering a contraceptive device according to FIG. 5 out a distal end thereof.

FIG. 8 is a cross-sectional view of a fallopian tube illustrating the deployed contraceptive device as shown in FIG. 5. The expandable material is shown in its fully expanded state.

FIG. 9 illustrates a graph of the expansion rate of the expandable material according to one embodiment.

FIG. 10 is a cross-sectional view of the female reproductive anatomy illustrating a deployed contraceptive device.

FIG. 11 is a graph illustrating the short-term and long-term contraceptive efficacy of the device according to one embodiment.

### DETAILED DESCRIPTION OF THE PREFERRED EMBODIMENTS

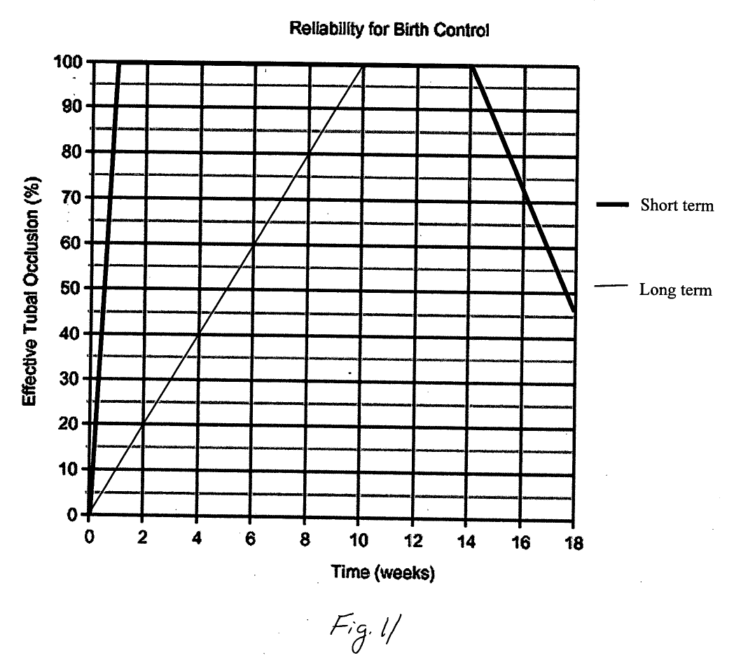

FIG. 1 illustrates an embodiment of a contraceptive device 2 for placement within a fallopian tube 100 (as shown in FIG. 10) of a subject. The contraceptive device 2 includes distal anchor member 4, a proximal anchor member 6, and an elongate connecting member 8. The distal anchor member 4, proximal anchor member 6, and elongate connecting member 8 are preferably expandable from a first, constrained configuration to a second, relaxed configuration. For example, the contraceptive device 2 is formed of one or more resilient materials that permit the device 2 to expand outwardly in the axial and/or radial directions upon release of a constraining force. Illustrative resilient materials usable in connection with the contraceptive device 2 include, for example: platinum; copper; stainless steel; shape memory metals such as nickel/titanium alloys such as NITINOL and TINEL; copper/zinc/aluminum alloys; copper/aluminum/nickel alloys; silver/cadmium alloys; gold/cadmium alloys; copper/tin alloys; copper/zinc alloys; indium/titanium alloys; nickel/aluminum alloys; iron/platinum alloys; manganese/copper alloys; iron/manganese/silicon alloys; cobalt/chromium alloys.

Still referring to FIG. 1, in one embodiment, the distal anchor member 4 and the proximal anchor member 6 are generally configured in a conical shape. For example, as seen in FIG. 1, the most proximal end 6a of the proximal anchor member 6 is configured as a coil structure with a decreasing radius of curvature until the elongate connecting member 8 is reached. Similarly, the most distal end 8a of the distal anchor member 8 is configured as a coil structure with a decreasing radius of curvature until the elongate connecting member 8 is reached. The distal most end 8a may be formed as an atraumatic tip to facilitate deployment within the fallopian tube 100.

In one aspect of the invention, the proximal and distal anchor members 6, 8 as well as the elongate connecting member 8 are formed as a unitary structure. For example, the contraceptive device 2 may take the form a single elongate member such as, for instance, a wire. The contraceptive device 2 may include one or more radiopaque markers 10 to aid in fluoroscopic placement. For example, a radiopaque substance such as gold or tantalum may be disposed on or integrated into the contraceptive device 2. Of course, radiopaque markers 10 may be omitted if the device 2 is delivered using hysteroscopic guidance and suitable visual markers added such as a marker with distinct color or a different caliber wire at the point to mark the appropriate location on the wire.

As best seen in FIG. 1, in one embodiment, the proximal end 6a of the proximal anchor member 6 includes an attachment member 12. The attachment member 12 is used to releasable attach the contraceptive device 2 to a pusher member (discussed in detail below). The attachment member 12 may take the form of a loop, hook, other interlocking structure. This serves to maintain control of the device 2 until it is deemed in appropriate position. If the position of the unsheathed device 2 is not optimal, the pusher member may be used to provide backward force on the expanded device 2 while the delivery member such as a delivery catheter is pushed back over the device 2, thereby recovering the device 2 within the catheter. This feature permits the device 2 to be repositioned to an optimal site within the fallopian tube.

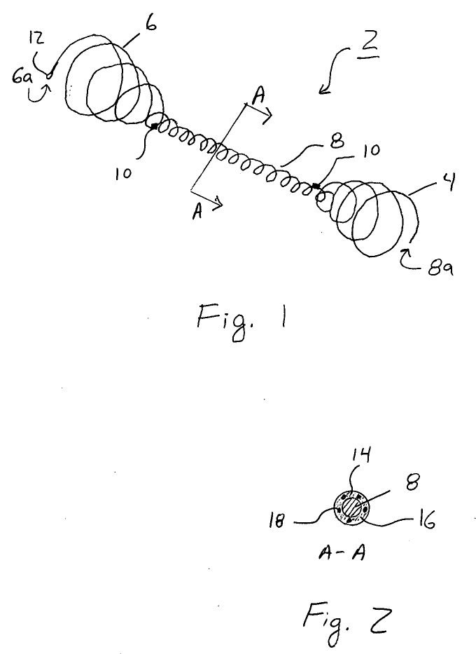

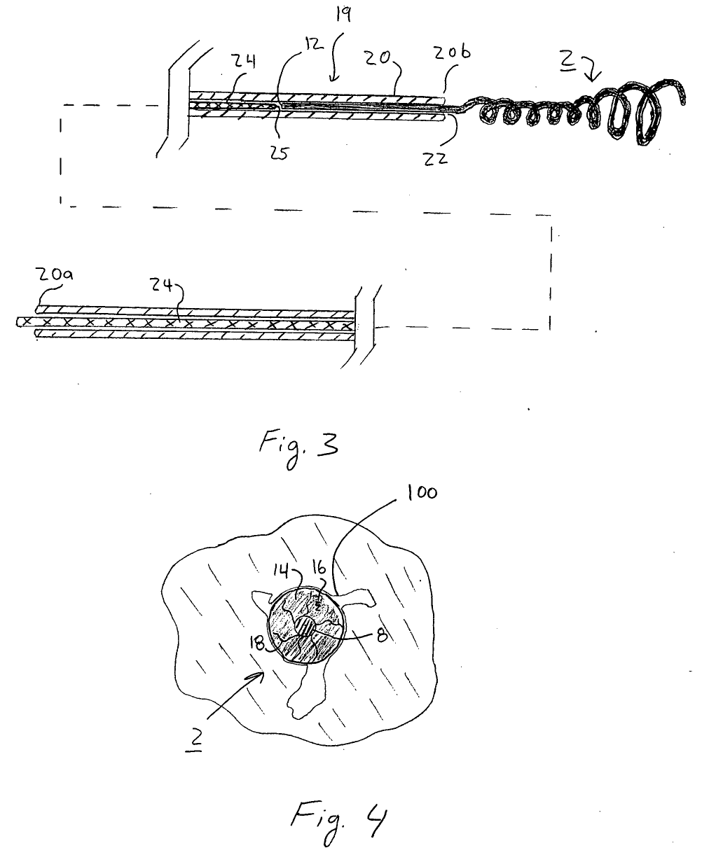

With reference to FIG. 2, the contraceptive device 2 advantageously includes an expandable material 14 disposed on at least a portion of the contraceptive device 2. The expandable material 14 is preferably loaded or coated on the contraceptive device 2 in a substantially un-expanded state (e.g., a dry state). The expandable material 14 preferably expands in response to contact with the fallopian tube 100. For example, expansion may initiate in response to the aqueous environment of the fallopian tube 100. Other triggers that may be used to initiate expansion include temperature, pH, salinity, osmolarity, or osmolality.

In one preferred aspect of the invention, the expandable material 14 is formed from a hydrogel material. In one aspect of the invention, the expandable material 14 has a relatively slow rate of expansion. In this regard, it is possible to re-sheath or re-load the contraceptive device 2 within the delivery member (described below). For example, the expandable material 14 may expand or swell over a period of hours or days with full expansion reached with a time period of about 1 day to about 4 days.

In one embodiment, the expandable material 14 may be coated or loaded (for example, within pores or other interstitial space) with one or more drugs or therapeutic agents 16. Exemplary therapeutic agents include spermicidal agents, anti-viral agents, anti-microbial agents (e.g., antibiotics), sclerosing agents, and inflammatory agents. The therapeutic agents 16 may be loaded or otherwise disposed on or in the expandable material 14 to elute over a specified period of time. For example, the release kinetics of the therapeutic agents 16 may be adjusted for a particular therapeutic effect. In the case of a spermicidal agent (e.g., NONOXYNOL-9), spermicide may be released for a period of hours, days, or months to ensure contraceptive efficacy until the device 2 and/or expandable material 14 completely occludes the fallopian tube 100. Other agents which may be utilized include hormones or hormone combinations (e.g., progestin, progestin/estrogen combination) to provide birth control efficacy for a relatively short period of time (generally less than 3 or 4 months). The hormones may be combined with a spermicidal agent.

In one alternative embodiment, the expandable material 14 is formed from a biodegradable material that degrades or is absorbed by the body over a period of time. The expandable material 14 may degrade relatively slowly over time such that secondary occlusion of the fallopian tube 100 has occurred. Such secondary or long-term occlusion of the fallopian tube 100 may be caused by an inflammation/fibrosis inducing agent incorporated into the device 2. For example, the inflammation inducing agent may include quinacrine, tetracycline, erythromycin, or bleomycin. In one aspect of the device 2, fibrous material 18 is incorporated into the contraceptive device 2 to provoke low-level, long term fibrosis of the fallopian tube 100. For example, polyethylene terephthalate (PET) (e.g., DACRON) or other fibrous material 18 may be secured to the contraceptive device 2 through a mechanical attachment (e.g., braided) or though the use of an adhesive material.

In another aspect, the fibrous material 18 may be placed over the expandable material 14. For example, the fibrous material 18 may take the form of an outer woven covering or jacket that permits fluid ingress/egress. Typically, a majority of the expansion of the expandable material 14 may take place within the first 24 hours after placement. Maximum expansion is reached several days later (e.g., 4-5 days post-placement).

In one embodiment, the expandable material 14 may be at least partially contained in an outer restraint or shell such as, for example, a silicone shell to reduce the expansion rate of the expandable material 14 to facilitate placement and repositioning if necessary.

FIG. 2 illustrates an enlarged cross-sectional view of the contraceptive device 2 of FIG. 1. The elongate connecting member 8 may be formed from a central core member or wire. An expandable material 14, for example, a hydrogel is coated on the exterior of the elongate connecting member 8. As seen in FIG. 2, the expandable material 14 is in the un-expanded state, for example, prior to deployment. A fibrous material 18 is also embedded within or disposed on the expandable material 14.

In another embodiment, the expandable material 14 is placed on the device 2 (e.g., core member 26 as shown in FIGS. 5 and 6) and a fibrous material 18 is placed around the device 2 (e.g., core member 26) such that when the device 2 expands, the fibrous material 18 abuts against an inner surface of the fallopian tube. The fibrous material 18 and expandable material 18 are, however, porous enough to permit extensive ingrowth of fibrosis into the interstices of the device 2 to cause effective tubal occlusion.

In still another aspect, the expandable material 14 is placed in discrete or segmented regions of the device 2 (e.g., core member as shown in FIGS. 5 and 6). Those areas not covered by the expandable material 14 have fibrous material 18 (e.g., woven polyester fibers) such that when the expandable material 14 swells or expands, the fibrous material 18 abuts against an inner surface of the fallopian tube and allows extensive ingrowth of fibrosis into the interstitial spaces of the device 2 to cause effective tubal occlusion.

The expandable material 14 may also be coated with an optional material which limits ingress of water to thereby prevent or delay expansion of the expandable material 14. In one embodiment, the expandable material 14 may degrade slowly over time (e.g., 12 to 36 hours post-placement) to allow diffusion of water to further expand the material 14.

FIG. 3 illustrates a device 19 for placement of the contraceptive device 2 within a fallopian tube 100. The device 19 includes a delivery member 20 having a lumen 22 therein that passes from a proximal end 20a to a distal end 20b. The delivery member 20 may take the form of a microcatheter or the like. A pusher member 24 is slidably disposed within the lumen 22 of the delivery member 20. One or both of the delivery member lumen 22 and pusher member 24 may have a lubricous coating to aid the pushability of the pusher member 24 within the lumen 22. The pusher member 24 may include a mating attachment member 25 on a distal end thereof that engages with the attachment member 12 on the contraceptive device 2.

For deployment, contraceptive device 2 is first loaded into the lumen 22 of the delivery member 20. In one embodiment, the contraceptive device 2 is loaded into (e.g., pulled into) a peel-away sheath (not shown). The sheathed contraceptive device 2 may then be inserted into a proximal end 20a of the delivery member (e.g., hub of microcatheter). Once the device 2 is advanced distally enough within the delivery member 20, the peel-away sheath is removed and the device 2 is advanced distally by distal advancement of the pusher member 24. Advancement of the device 2 may be monitored in real-time using fluoroscopic visualization. Once the contraception device 2 is advanced distally within the delivery member 20, its position may be verified by, for example, contrast injection into a cervical cannula side port (not shown). For delivery, the contraceptive device 2 is held in place using the pusher member 24 while the outer delivery member 20 (e.g., sheath) is retracted in the proximal direction to un-sheath the contraceptive device 2 as is shown in FIG. 3.

If the physician determines that the placement of the contraceptive device 2 is acceptable, a release mechanism may be activated to release the contraceptive device 2 from the attachment member 12 on the pusher member 24. This may involve, for example, a rotation or torquing of the pusher member 24 to release disengage the contraceptive device 2 from the pusher member 24. If the positioning is not acceptable, the contraceptive device 2 may be withdrawn proximally within the delivery member 20 by retraction of the pusher member 24 in the proximal direction. Another attempt may then be made to deploy the contraceptive device 2.

After deployment of the contraception device 2, a repeat contrast injection may be delivered to image the final placement of the device 2. Another contraception device 2 may then be placed in the opposing or contralateral fallopian tube 102 (as shown in FIG. 10).

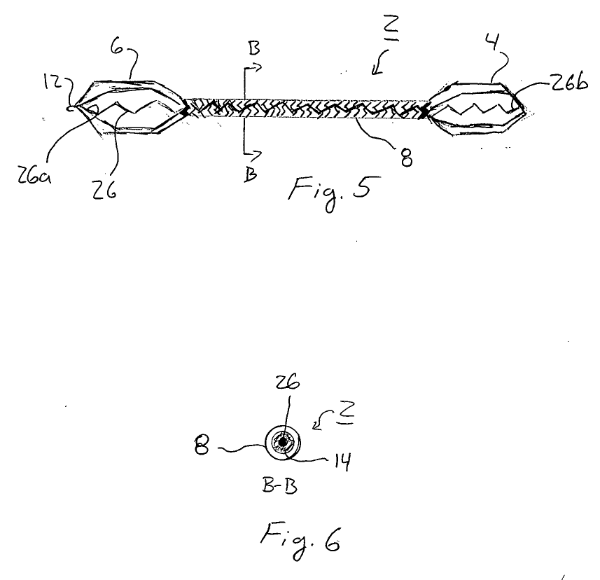

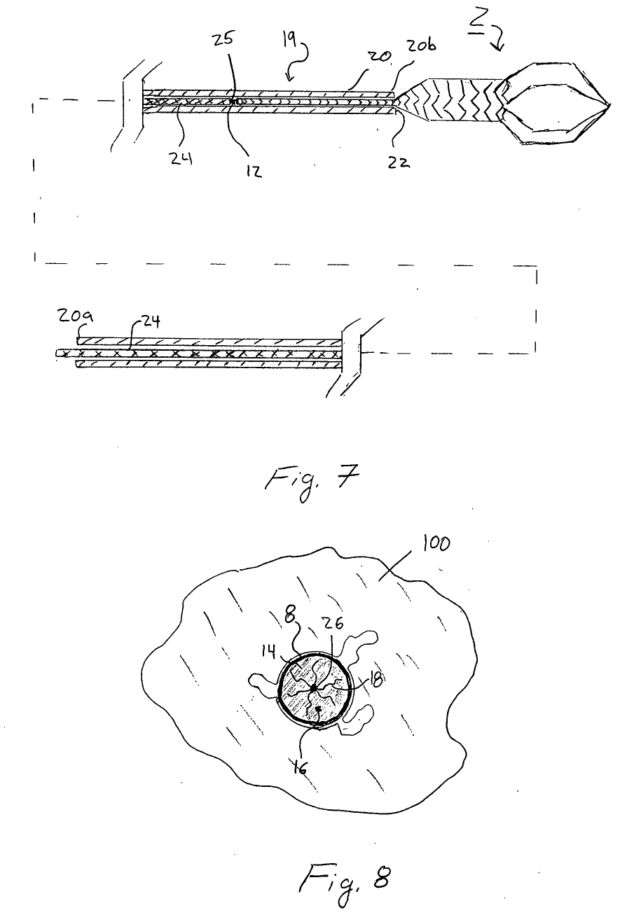

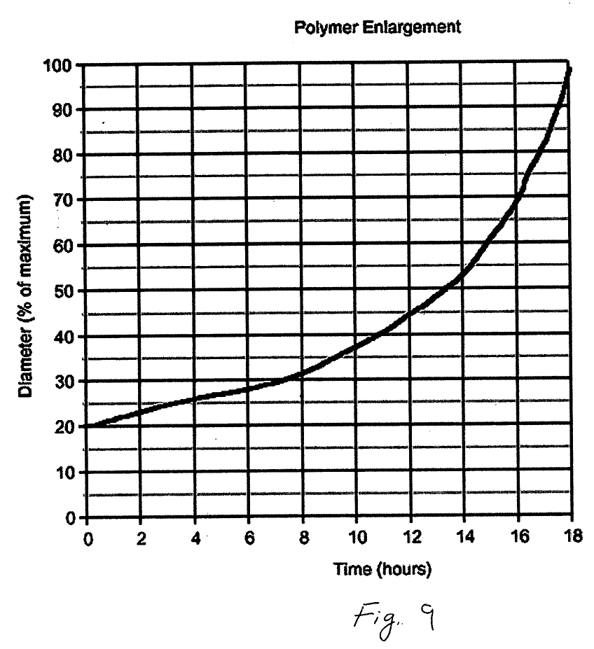

FIG. 4 illustrates a cross-sectional view of the contraception device 2 deployed within a fallopian tube 100. As seen in FIG. 4, the expandable material 14 surrounding the elongate connecting member 8 has expanded to its fully expanded state, thereby completely occluding the fallopian tube 100. Fibrous material 18 is interspersed within the expandable material 14 to promote longer-term fibrosis and/or inflammation within the fallopian tube 100.

The contraception device 2 generally operates as a two-stage occlusion device. The first stage of occlusion is due substantially to the expansion of the expandable material 14 located on the occlusion device 2. This first stage may be accompanied by one or more eluting therapeutic agents 16 that facilitate contraceptive efficacy (e.g., spermicidal agents, inflammation agents, etc.). The second stage of occlusion is long-term and caused by low-level, yet long-term inflammation/fibrosis. Generally, the first stage of occlusion is such that complete or substantially complete occlusion occurs within about four days of delivery of the occlusion device 2. Complete occlusion may occur within one day of deployment. The second stage of occlusion generally initiates with a few weeks to months after delivery and provides for long-term occlusion of the fallopian tube 100.

As stated above, the expandable material 14 may include one or more therapeutic agents 16 to supplement or aid in the contraceptive abilities of the device 2 prior to the second stage of occlusion. In this regard, the contraceptive device 2, once deployed, provides immediate or near immediate contraceptive abilities. The contraceptive device 2 does not require follow-up visits to a physician to confirm occlusion of the fallopian tube(s). This provides a significant benefit over past devices which require follow-up confirmation HSG tests.

FIG. 5 illustrates an alternative embodiment of the contraceptive device 2. The contraceptive device 2 includes a distal anchor member 4, a proximal anchor member 6, and an elongate connecting member 8. The proximal and distal anchor members 4, 6 may be formed as expandable cage or scaffolding structure. For example, the proximal and distal anchor members 4, 6 may be expandable into three-dimensional polyhedral structures as illustrated in FIGS. 5, 7, and 9. The elongate connecting member 8 may be formed as an expandable stent or similar structure. As seen in FIG. 5, the contraceptive device 2 may include a core member 26 disposed centrally, within a lumen of the expandable elongate member 8 and connects at a proximal end 26a to the proximal anchor member 6 and at a distal end 26b to the distal anchor member 4. The core member 26 may be expandable in the radial and axial directions. For example, the core member 26 may have a serpentine or zig-zag shape.

FIG. 6 illustrates a cross-sectional view of the contraceptive device 2 taken along the line B-B in FIG. 5. The core member 26 is surrounded by an expandable material 14 of the type disclosed herein. The core member 26 and expandable material 14 are located generally within the lumen of the elongate connecting member 8 (e.g., a stent structure).

In an alternative aspect, at least a portion of the contraceptive device 2 may be coated or otherwise loaded with a material that promotes the growth of fibroblasts or myofibroblasts to induce more rapid fibrosis (and thus closure) of the fallopian tube. Exemplary materials include connective tissue growth factor, lactate glycolic acid polymers, or similar cellular promoters.

FIG. 7 illustrates another embodiment of a device for placement of the contraceptive device 2 illustrated in FIGS. 5 and 6 within a fallopian tube 100. The device includes a delivery member 20 having a lumen 22 therein that passes from a proximal end 20a to a distal end 20b. As in the prior embodiment, the delivery member 20 may take the form of a catheter (e.g., a microcatheter). A pusher member 24 is slidably disposed within the lumen 22 of the delivery member 20. One or both of the delivery member lumen 22 and pusher member 24 may have a lubricous coating to aid the pushability of the pusher member 24 within the lumen 22. The pusher member 24 may include a mating attachment member 25 on a distal end thereof that engages with the attachment member 12 on the contraceptive device 2.

The contraceptive device 2 may be loaded into the delivery member lumen 22 as described above. As seen in FIG. 7, the distal anchor member 4 and a portion of the elongate connecting member 8 are ejected from the distal end of the delivery member 20. In this regard, the contraceptive device 2 is self-expanding from a constrained configuration (inside the delivery member 20) to a relaxed, expanded configuration. The contraceptive device 2 may be ejected by retracting the outer delivery member 20 keeping the contraceptive device 2 substantially stationary using the pusher member 24. Detachment of the contraceptive device 2 may be accomplished disengaging the mating attachment member 25 from the attachment member 12. This may be accomplished by, for example, twisting or torquing the pusher member 24. Alternatively, a trigger (not shown) or other disengagement mechanism commonly known to those skilled in the art may be utilized to effectuate separation between the contraceptive device 2 and the pusher member 24.

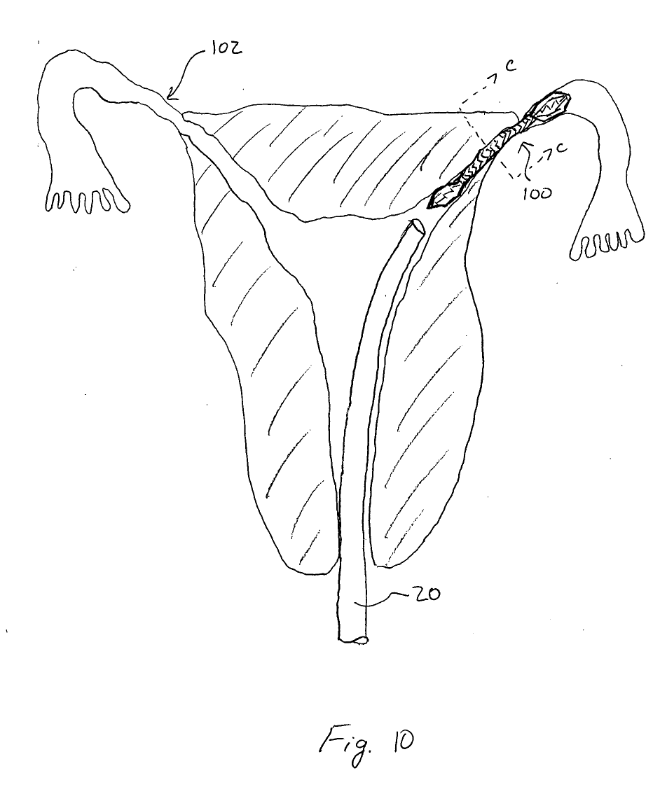

FIG. 8 illustrates a cross-sectional view of a fallopian tube 2 having the contraceptive device 2 deployed therein (e.g., taken along the line C-C in FIG. 10). The elongate connecting member 8 is shown in an expanded state such that all or a portion of the external surface of the elongate connecting member 8 abuts against an internal surface of the fallopian tube 100. In addition, the expandable material 14 is shown in its fully expanded state such that it completely or nearly completely occludes the fallopian tube 100. A fibrous material 18 is shown being interspersed in the expandable material 14. One or more therapeutic agents 16 may be located on or within the expandable material 14 as described in detail herein.

FIG. 9 illustrates a graph illustrating the expansion rate of the expandable material 14 according to one aspect of the invention. As seen in FIG. 9, the initial rate of expansion starts off slowly. In this regard, the device 2 is able to be repositioned if the initial placement of the device 2 is less than desired. The rate of expansion progressively increases until maximum expansion is reached at around 18 hours post-placement.

FIG. 10 illustrates a cross-sectional view of the female anatomy showing the contraceptive device 2 deployed within the fallopian tube 100. In one preferred aspect of the invention, the contraceptive device 2 is placed in the isthmic region of the fallopian tube 100 as is shown in FIG. 10. After a first contraceptive device 2 has been placed in one fallopian tube 100, a second contraceptive device 2 may be placed in the second fallopian tube 102 (e.g. contralateral side fallopian tube).

With reference to the devices 2 illustrated in FIGS. 1 and 5, when the device 2 is in the relaxed or expanded configuration, the distal and proximal anchor members 4, 6 may have an outer diameter between about 2 mm and about 5 mm. Moreover, the distal and proximal anchor members 4, 6 may have a length from around 0.5 cm to around 1.5 cm. The elongate connecting member 8 (in its expanded state) may have a length from around 3.0 cm to 5 cm and a width from around 1.5 mm to 4 mm. Of course, other dimensions and geometries are intended to fall within the scope of the invention recited herein.

With regard to delivery of the device 2, the procedure may be performed in the window of day 7 through 14 of the patient's menstrual cycle to limit the chance of a luteal phase pregnancy. If a patient is on long-term contraceptive such as Depo-Provera, the procedure may be performed at any time. A qualitative urine hCG test may be performed on the day of the procedure and the negative result is documented on the patient's chart. The patient may receive intravenous conscious sedation with midazolam and fentanyl titrated to effect by a certified intravenous conscious sedation nurse or receive only a local anesthetic around the cervix (para-cervical block) placed by the physician performing the procedure. A non-steroidal anti-inflammatory agent such as thirty milligrams of intravenous ketorolac and an antibiotic such as 1.0 g of intravenous ceftriaxone may be administered before the procedure is started. The patient may be placed in the lithotomy position in stirrups on, for example, an angiography or fluoroscopy table. The vulvar and perineal areas are prepped and draped in usual sterile fashion with BETADINE solution. A sterile metal speculum or disposable plastic speculum may then be placed in the vaginal vault and the cervix is identified.

Both the vaginal vault and cervix may be painted with a BETADINE paint solution. The cervical os may be cannulated with a catheter which provides both a working port with inner diameter from 2 French to 6 French and a side arm port to inject contrast material into the uterus. This catheter may or may not have balloons (proximal and/or distal to the cervix) to facilitate contrast retention during the procedure. This would include a catheter such as a 12-F balloon cervical cannula (available from Cook, Inc., Bloomington, Ind.). The internal balloon is inflated to achieve a seal. A cervical tenaculum may be used if needed for traction on the uterus. A contrast material such as VISIPAQUE 320 (Amersham Health, Princeton, N.J.) may be injected through the side port (not shown) of the delivery member 20 for the hysterosalpingography (HSG). A 5-F Kumpe catheter (Cook) can be used for selective salpingograms. After identification of the fallopian tubes 100, 102, one fallopian tube (e.g., fallopian tube 100) is selected for initial placement. Through the cervical cannula, with or without the 5-F catheter in place, the delivery member 20 (e.g., a microcatheter between 1.5 and 6 F) may be tracked over a hydrophilic guidewire located distally in the fallopian tube 100. The delivery member 20 may take the form of a single lumen catheter having an outer diameter sized within the range from 1.5 F to 6 F. The catheter may be tapered (with a larger proximal end and smaller distal end) or non-tapered. The exterior or outer coating may consist of hydrophilic or hydrophobic compounds or any combination thereof.

In one aspect, the delivery member 20 (e.g., catheter) may include one or more radiopaque markers (gold, tantalum, etc.) to aid in placement of the device 2. The catheter may have a lumen for receiving a guiding member such as, for example, a guide wire. For example, the catheter may have a lumen with an inner diameter within the range from 0.005 inches (0.127 mm) to 0.0394 inches (1 mm). The catheter is placed over a guide wire which may range in size from around 0.13 mm to 0.99 mm. Typically, the guide wire is placed through the hub of the catheter (in the proximal end) and advanced until the tip of the guide wire emerges from the distal tip of the catheter.

The wire is then removed and contrast injected through the delivery member 20 to verify the placement of the delivery member 20 in the lumen of the fallopian tube 100. Once intraluminal placement is verified, the device 2 is prepared. The device 2 is hooked to the pusher member 24 and pulled into a constraining peel-away sheath (not shown). The peel-away sheath is used to load the constrained contraceptive device 2 into the hub of the delivery member 20. Once the device 2 is advanced into the delivery member 20 distally enough, the peel-away sheath is removed and the pusher member 24 advanced distally.

Distal advancement of the device 2 may take place under real-time fluoroscopic visualization. Once the device 2 is in place distally in the delivery member 20 and the position is verified, for example, via a contrast injection into a cervical cannula side port, the contraceptive device 2 may be held in place and the delivery member 20 is pulled back towards the physician in the proximal direction to un-sheath the device 2. If the physician determines the device 2 placement is acceptable, the release mechanism is activated and the contraceptive device 2 is fully deployed. The delivery member 20 is then pulled back into the cannula and a repeat contrast injection performed for final placement image. The fallopian tube 102 on the contralateral side is then selected and the device 2 is deployed as described previously. The patient may be monitored by a nurse post-procedure.

FIG. 11 is a graph illustrating the short-term and long-term contraceptive efficacy of the device 2 according to one aspect of the invention. Short-term contraceptive efficacy is achieved within about one week of deployment of the device 2. Short-term contraceptive efficacy is achieved primarily through expansion of the expandable material 14 and/or elution of the drug(s) or therapeutic agent(s). Long-term contraceptive efficacy is achieved within about ten weeks of deployment of the device 2. The long-term contraceptive efficacy is achieved, for example, through fibrosis of the fallopian tube. After long-term contraceptive efficacy has been established, the contraceptive efficacy provided by the expandable material 14 and/or elution of the drug(s) or therapeutic agent(s) begins to decrease. This may be due to, for example, degradation or absorption of the expandable material 14. While the short-term contraceptive efficacy decreases, the overall contraceptive efficacy of the device 2 remains high because of the longer-term occlusion of the fallopian tubes. In this regard, immediate or near-immediate occlusion of the fallopian tube is achieved upon delivery of the device 2.

While embodiments of the present invention have been shown and described, various modifications may be made without departing from the scope of the present invention. The invention, therefore, should not be limited, except to the following claims, and their equivalents.

What is claimed is:

1. A contraceptive device for placement in a fallopian tube comprising:
an expanding distal anchor member;
an expanding proximal anchor member;
an expandable elongate member connecting the distal anchor member to the proximal anchor member; and
an expandable material disposed on at least a portion of the contraceptive device, whereupon delivery of the contraceptive device, the expandable material expands to completely occlude the fallopian tube.

2. The device of claim 1, wherein the expandable material comprises a hydrogel.

3. The device of claim 1, wherein the expandable material is loaded with a material selected from the group consisting of a spermicidal agent, an anti-viral agent, an anti-microbial agent, a sclerosing agent, and an inflammatory agent.

4. The device of claim 1, wherein the expandable material comprises a biodegradable material.

5. The device of claim 3, wherein the inflammatory agent comprises a fibrous material.

6. The device of claim 1, wherein the fallopian tube is completely occluded within four days after delivery of the contraceptive device.

7. The device of claim 1, further comprising an attachment member located on the proximal anchor member.

8. The device of claim 1, further comprising one or more radiopaque markers disposed on the contraceptive device.

9. The device of claim 1, further comprising a core member disposed within a lumen of the expandable elongate member and connected at a proximal end to the proximal anchor member and connected at the distal end to the distal anchor member.

10. An apparatus for placement of a contraceptive device in a fallopian tube comprising:
a delivery member having a lumen passing from a proximal end to a distal end;
a contraceptive device adapted for disposal within the lumen of the delivery member, the contraceptive device comprising:
an expanding distal anchor member;
an expanding proximal anchor member;
an expandable elongate member connecting the distal anchor member to the proximal anchor member;
an expandable material disposed on at least a portion of the contraceptive device, whereupon delivery of the contraceptive device, the expandable material expands to completely occlude the fallopian tube; and
a pusher member slidable within the lumen of the delivery member, the pusher member being located proximal of the contraceptive device.

11. The apparatus of claim 10, further comprising an attachment member located on the proximal anchor member and a mating attachment member located on a distal end of the pusher member, wherein the mating attachment member located on the distal end of the pusher member is detachable from the attachment member located on the proximal anchor member.

12. The apparatus of claim 10, wherein the expandable material comprises a hydrogel.

13. The apparatus of claim 10, wherein the expandable material is loaded with a material selected from the group consisting of a spermicidal agent, an anti-viral agent, an anti-microbial agent, a sclerosing agent, a hormone agent, and an inflammatory agent.

14. The apparatus of claim 10, wherein the fallopian tube is completely occluded within four days after delivery of the contraceptive device.

15. A method of occluding a fallopian tube comprising the steps of:
providing a contraceptive device, the device comprising:
an expanding distal anchor member;
an expanding proximal anchor member;
an expandable elongate member connecting the distal anchor member to the proximal anchor member; and
an expandable material disposed on at least a portion of the contraceptive device;
inserting the contraceptive device into the fallopian tube, whereupon insertion, one or both of the distal and proximal anchor members expand to secure the contraceptive device to an interior surface of the fallopian tube; and
expanding the expandable material to completely occlude the fallopian tube.

16. The method of claim 15, wherein the fallopian tube is completely occluded within four days after delivery of the contraceptive device.

17. The method of claim 15, wherein the fallopian tube is completely occluded within one day after delivery of the contraceptive device.

18. The method of claim 15, further comprising the step of releasing an agent loaded in the expandable material, the agent being selected from the group consisting of a spermicidal agent, an anti-viral agent, an anti-microbial agent, a sclerosing agent, a hormone agent, and an inflammatory agent.

19. The method of claim 15, wherein the expandable material is substantially degraded after a month or more.

20. The method of claim 19, wherein the fallopian tube remains completely occluded after degradation of the expandable material.
